(19) United States
(12) Patent Application Publication
(10) Pub. No.: US 2017/0273704 A1
(43) Pub. Date: Sep. 28, 2017

(54) COMBINED TWEEZERS AND METHOD OF USING COMBINED TWEEZERS

(71) Applicant: Children's Medical Center Corporation, Boston, MA (US)

(72) Inventor: Liu Kaifeng, Boston, MA (US)

(21) Appl. No.: 15/508,720

(22) PCT Filed: Sep. 4, 2015
(86) PCT No.: PCT/US2015/048534

§ 371 Date: Mar. 3, 2017

Related U.S. Application Data
(60) Provisional application No. 62/046,715, filed on Sep. 5, 2014.

(51) Int. Cl. A61B 17/30

(52) U.S. Cl. CPC A61B 17/30; A61B 2017/00424

(57) ABSTRACT

Disclosed is a medical device that is capable of grasping multiple sites of one object or multiple objects simultaneously. The medical device includes a platform and a plurality of pincer sets that are attached to the platform. The plurality of pincer sets extend from the platform at varied lengths and at individually operable. Accordingly, a plurality of surgical sites are grasped simultaneously and independently using multiple pincer sets.

The present application claims the benefit of U.S. provisional application No. 62/046,715 filed Sep. 5, 2014, which is incorporated herein by reference in its entirety.

### BACKGROUND

(a) Technical Field

The present invention relates to combined tweezers, and more specifically, relates to multiple pincer tweezers that allow multiple surgical areas to be grasped simultaneously with improved precision.

(b) Background Art

Conventional tweezers include a set of pincers that are used to grasp one site or one object precisely. Particularly, a user typically holds a pair of tweezers with a thumb on one arm and index and middle fingers on the other arm to operate the tweezers stably. In addition, such conventional tweezers are often used today as a medical device for surgical purposes such as vessel cannulation. When a surgeon or operator of a medical device is required to grasp multiple sites of, for example, a vessel opening, either multiple devices are required or extra assistance is required from another person, thus, increasing the difficulty and time of accessing the vessel. For example, a surgeon may grasp an object (e.g., a vessel, tissue, etc.) with the tweezers and may be required to operate another medical device with the other hand. Thus, an assistant may be required to operate a second pair of tweezers to, for example, move the second object toward the first object to allow the surgeon to operate the other medical device.

In addition, multiple conventional tweezers have been attempted to be operated simultaneously by one hand, as shown in FIG. 1. Particularly, FIG. 1 shows a user attempting to simultaneously operate two independent tweezers 100, 105 with one hand. In particular, FIG. 1 shows a grip of a thumb on one arm of both tweezers and a middle finger one second arm and an index finger one the other second arm (e.g., one on each tweezer). However, as known by those skilled in the art, such operation is unsteady and decreases the precision of a surgical procedure. Accordingly, it is desirable to provide a medical device capable of grasping multiple objects and allowing a surgeon to operate a second medical device simultaneously with another hand without the need of extra assistance.

### SUMMARY

The present invention provides a combined tweezer system that allows multiple surgical areas to be grasped simultaneously using multiple pincer sets. Accordingly, medical device stability and precision may be improved.

In particular, according to an exemplary embodiment of the present invention, a medical device is provided that includes a platform and a plurality of pincer sets attached to the platform and extending from the platform at different lengths. The plurality of pincer sets may be individually operable and may be attached to the platform via a weld, a mold, an adhesive, a fastening mechanism, a magnet, or the like. The platform may be flat, angled, spatial, or may be a varied plane. Further, the platform may include a grip attached to a bottom of the platform that improves operation stability. The various pincer sets attached the platform may also be adjusted using a fastener (e.g., a screw mechanism) to either spread the arms of the pincer sets apart or narrow the space therebetween. Additionally, the fastener may be used to vary the height of the different pincer sets on the platform. Accordingly, the plurality of pincer sets may be operated independently and simultaneously using one hand, thus improving stability of the medical device.

Furthermore, according to another exemplary of the present invention, a medical device is provided that includes a pair of mutually opposed arms attached at a first end of each arm; a first pair of pincers, separated by a first distance and extending from a second end of each arm; and a second pair of pincers, separated by a second distance, with individual pincers attached to each mutually opposed arm. The first distance and the second distance may be different from each other. The distance between the two set of pincers allows one set of pincers to be closed at a different time than the other and thus, objects may be grasped independently. Alternatively, the distances between the pincer sets may be about the same and one of the pincer sets may include more than one tip, thus providing an additional grasping method.

Other aspects of the invention are disclosed infra.

### BRIEF DESCRIPTION OF THE DRAWINGS

The disclosure will hereinafter be described with reference to the accompanying drawings, wherein like reference numerals denote like elements, and:

FIG. 1 illustrates an exemplary view of a plurality of tweezers operated independently according to the related art;

FIGS. 2A-2B illustrate an exemplary view of a plurality of pincer sets attached to a platform according to an exemplary embodiment of the present invention;

FIG. 3 illustrates an exemplary view of a plurality of pincer sets attached to a platform at varied lengths according to an exemplary embodiment of the present invention;

FIG. 4 illustrates an exemplary grip attached to a bottom of the platform according to an exemplary embodiment of the present invention;

FIG. 5 illustrates an exemplary fastener connected to the pincer sets according to an exemplary embodiment of the present invention;

FIG. 6 illustrates an exemplary view of multiple pincer sets connected to a single arm at varied distances according to an exemplary embodiment of the present invention;

FIG. 7 illustrates an exemplary view of the pincer sets of FIG. 6 operable independently according to an exemplary embodiment of the present invention;

FIG. 8 illustrates another exemplary view of the pincer sets of FIG. 6 operable independently according to an exemplary embodiment of the present invention;

FIG. 9 illustrates an exemplary view of multiple pincer sets connected to a single arm at the same distance according to another exemplary embodiment of the present invention;

FIG. 10 illustrates an exemplary view of varied pincer sets attached to a platform according to an exemplary embodiment of the present invention.

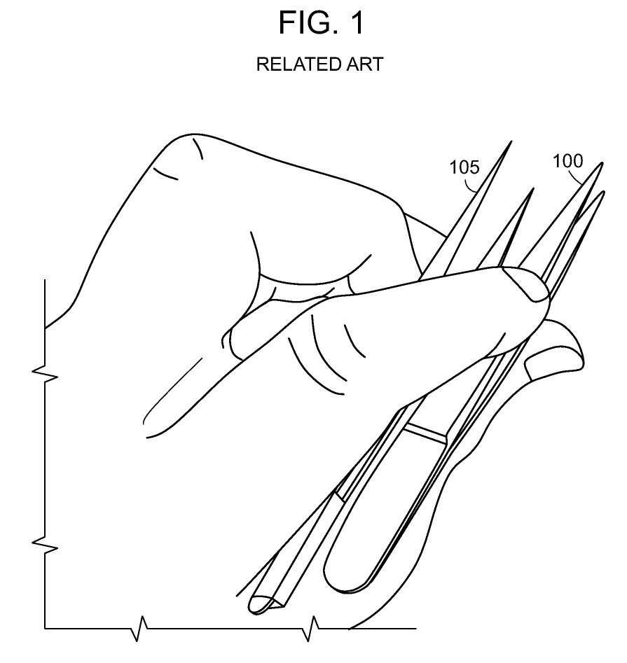

### DETAILED DESCRIPTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Combined tweezers are provided that allow multiple areas or objects to be grasped simultaneously to improve the stability of medical device operation and alleviate the need of additional assistance during medical procedures.

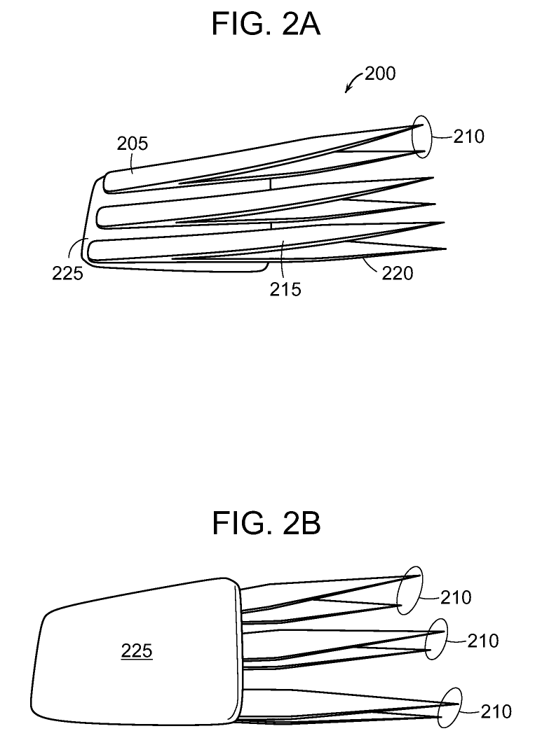

Referring now to the drawings, according to an exemplary embodiment of the present invention, various surgical sites may be grasped simultaneously by a medical device using one hand, as shown in FIGS. 2A-2B. In particular, as shown in FIG. 2A, a medical device 200 may include a platform 225 and a plurality of tweezers 205 each having at least one pincer set 210. Each tweezer 205 may include a first arm 215 and a second arm 220, ends of which together form a pincer set 210. Alternatively, the plurality of pincer sets 210 may be forceps, clips, or other related mechanisms. The plurality of pincer sets 210 may be attached to the platform using a fastening mechanism and may either extend from the platform at about the same length or at different lengths.

FIG. 2B shows an exemplary view of the platform 225 from a back view with the plurality of pincer sets 210 extending from the platform 225. Although the platform 225 in FIGS. 2A-2B is shown to be flat, the platform 225 is not limited thereto and may be flat, angled, spatial, varied plane, or the like. Although all attached to the platform in a similar manner, the plurality of pincer sets 210 may be operated individually. Accordingly, a surgeon may grasp the entire platform 225 and operate each set of tweezers 205 individually to grasp different surgical sites without requiring additional assistance and may therefore, simultaneously operate another medical device using the hand that is not operating the medical device 200 of the claimed invention.

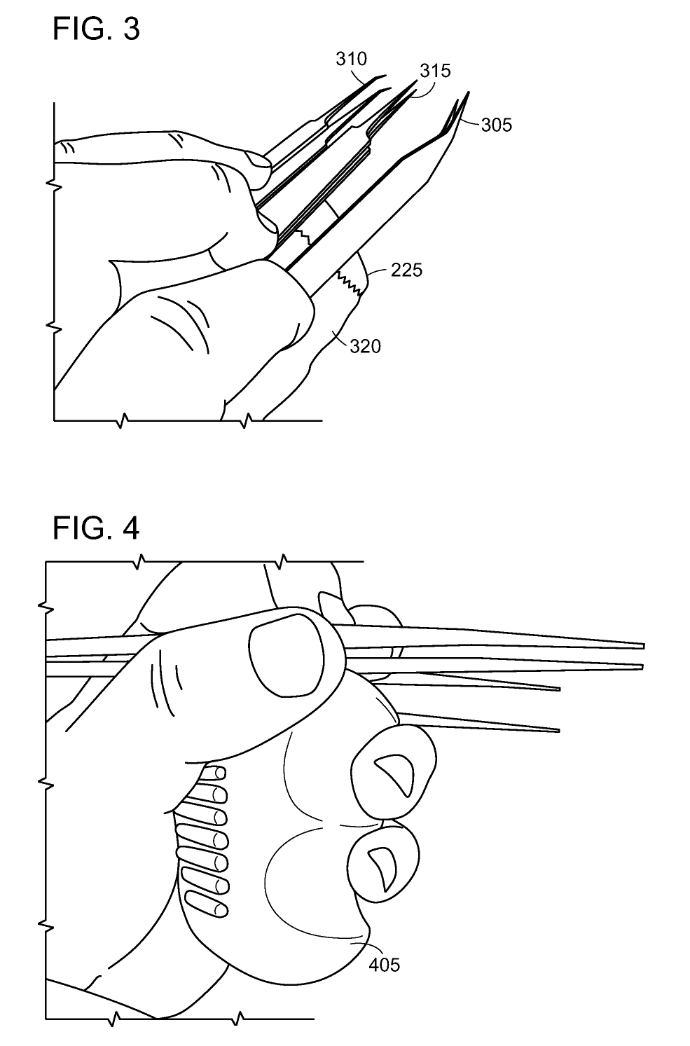

Further, as shown in FIG. 3, a plurality of pincer sets may be attached to the platform 225 via an adhesive. However, the invention is not limited thereto and the plurality of pincer sets may be attached to the platform 225 using various fastening mechanisms such as a weld, a mold, a magnet (e.g., magnetic field), or the like. In addition, the pincer sets may be attached differently to the platform. Particularly, as shown in FIG. 3, a first pincer set 305 may extemd a greater distance from the platform than a second pincer set 310 and a third pincer set 315. As an example, the three pincer sets (305, 310, 315) are shown to be disposed at three different lengths from the platform. The varied distances between the pincer sets allow different objects to be grasped or allow different areas of an object to be grasped independently at different times or simultaneously. In other words, the first pincer set 305 may be configured to grasp an object (e.g., a vessel, tissue, or the like) that is disposed deeper into a site than another object. Accordingly, while the first pincer set 305 is grasping a first object, the second or third pincer set 310, 315 may be used to grasp another object disposed at a distance from the first object. Thus, a surgeon may be able to grasp the different objects or areas while manipulating a different medical device in the other hand. In addition, since the pincer sets may be firmly secured to the platform via the various fastening mechanisms, stability of the device may be improved, thus increasing the precision of the surgical operation.

In another exemplary embodiment of the present invention, as shown in FIG. 4, a holder 405 may be attached to a bottom of the platform. The holder 405 may be formed as a grip to allow a user to more stably hold the grip while operating the pincer sets disposed on top of the platform. The holder may also be formed as an ergonomic grip to increase user comfort and stability. In detail, the holder may be gripped by a user's ring finger and pinky finger, a user's thumb may grasp an upper side of the holder while the index and middle finger operate the pincer sets.

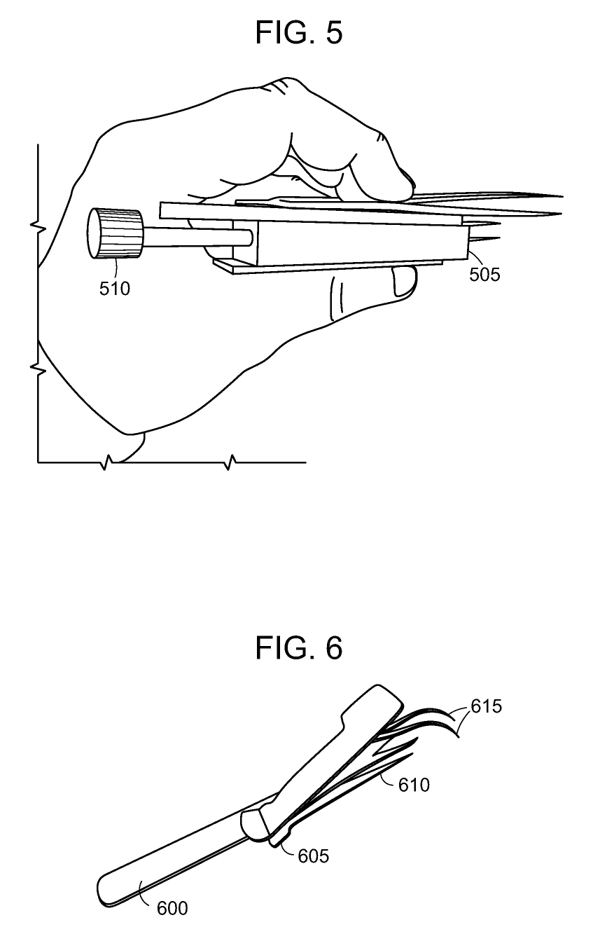

The claimed medical device may also include a fastener 505, as seen in FIG. 5. The fastener 500 may be connected at various locations on the platform and between the pincer sets to either vary the height of the pincer sets or vary the space between tips of each pincer set. In particular, the fastener may be connected between a pair of pincers to either increase or decrease the space between the pincers. The distance may be increased by moving the fastener 500 further from each pincer set or may be adjusted using a screw mechanism 510 attached to an end of the fastener 505. Additionally, the distance may be decreased or narrowed by moving the fastener 505 toward each pincer set. Alternatively, the fastener 505 may be attached at a bottom of one mutually opposed arm, as seen in FIG. 5. With such a configuration, the height of the different pincer sets may be adjusted on the platform. In addition, such a configuration may allow the angle of the pincer set to be varied to provide a platform with a plurality of pincer sets disposed at different angle to reach different surgical sites.

In another exemplary embodiment, at least one set of pincers may be operably connected to a second set of pincers to operate simultaneously at different positions. As shown in FIG. 6, the medical device 200 may include a pair of mutually opposed arms 605 attached at a first end of each arm. The mutually opposed arms 605 may be connected to an extending arm 600 which may be gripped by a user to operate the pincer sets. Further, a first pair of pincers 610 may be separated by a first distance and may extend from a second end of each arm. A second pair of pincers 615 may be separated by a second distance, different from the first distance, with individual pincers attached to each mutually opposed arm.

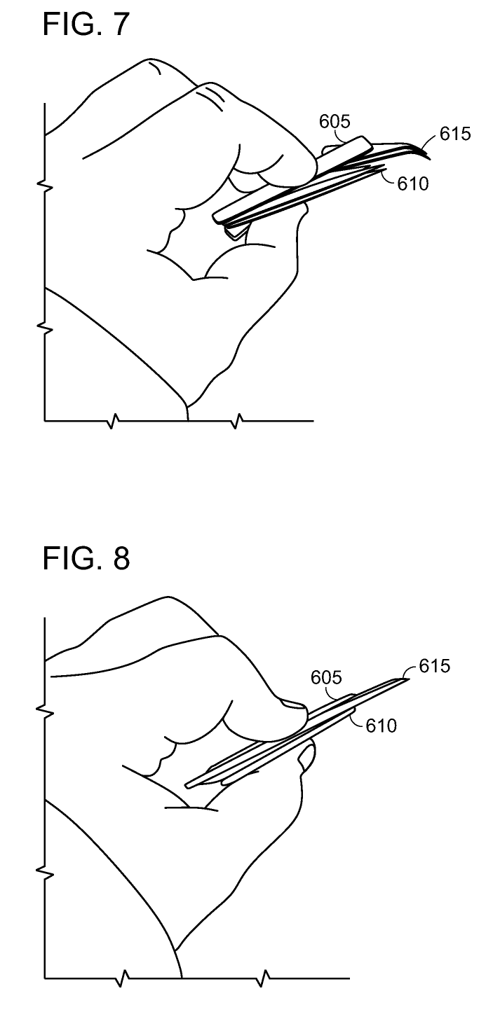

In particular, the second distance between the second pair of pincers may be less than the first distance between the first pair of pincers. The varied distance between the pincers allows for a delayed opening and closing of the device. In other words, the varied distance allows one object to be grasped before another. For example, as seen in FIG. 7, the second pair of pincers 615 may be closed in response to a user pressing down on the mutually opposed arms 605 while the first pair of pincers 610 remains open. Accordingly, a first object further away or at a different angle may be grasped before a second object. Further, FIG. 8 illustrates when both pair of pincers 610, 615 are closed in response to the mutually opposed arms 605 being pressed or squeezed together. In other words, the first pair of pincers 610 may be delayed from closing. To create the delayed opening and closing of the different pincer sets, one of the pairs of pincers may be made of a more resilient material and less rigid than the other pair of pincers. Thus, as an increased amount of pressure is applied to the mutually opposed arms 605, the first pair of pincers 610 may close on an object and may open upon release of such pressure, prior to the second pair of pincers 615 opening.

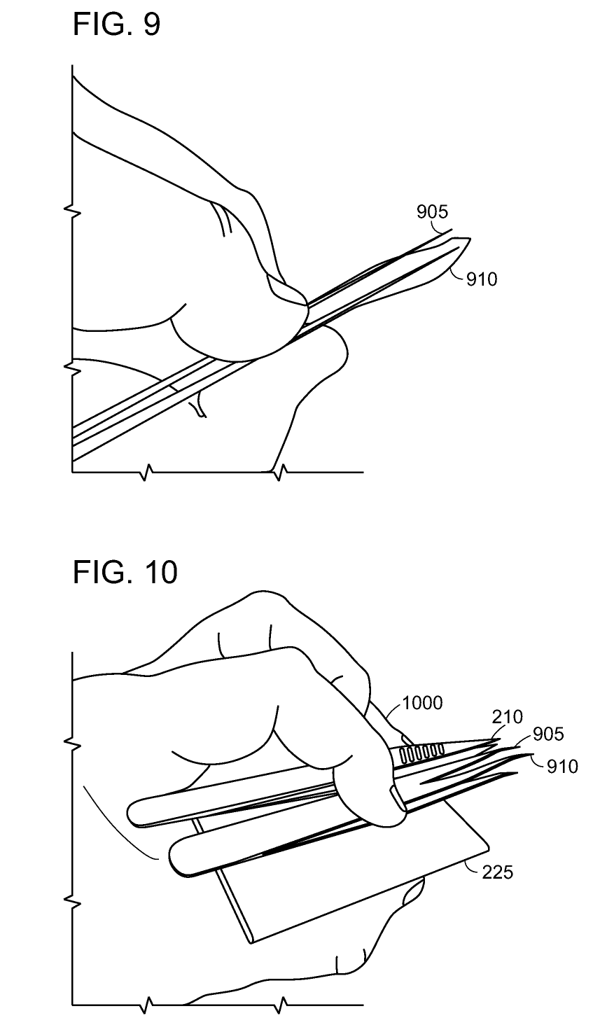

The pincer sets, according to another exemplary embodiment of the present invention, may also be formed as bi-pincer sets, as shown in FIG. 9. In particular, two different pincer sets 905, 910 may extend in a substantially parallel manner from one end of mutually opposed arms in the same direction. The pincer sets may be formed as different shapes to cause the pincer sets to operate individually. For example, as seen in FIG. 9 the first pincer set 905 may be formed of two substantially straight members and the second pincer set 910 may be formed of two bent members. The bent shape of the second pincer set 910 causes the second pincer set 910 to close before the first pincer set 905 in response to pressure being applied to the mutually opposed arms. Accordingly, a surgeon may be able to grasp two objects or sites independently without requiring two separate medical devices formed with pincer sets. For example, the second pincer set 910 may be closed down on a vessel and the vessel may be pulled toward another vessel that may then be grasped by the first pincer set 905 as an increased amount of pressure is exerted on the mutually opposed arms.

Moreover, FIG. 10 shows an exemplary view of a medical device 1000 including different pincer sets as described herein attached the platform 225. In particular, the medical device 1000 includes one pair of pincers 210 and a pair of bi-pincers 905, 910. The pair of bi-pincers may be, as described with reference to FIG. 9, a pair of mutually opposed arms with two separate pincer sets 905, 910 extending therefrom. In other words, the bi-pincers may be a pair of tweezers that includes to differently formed tips. A user may thus grasp the medical device and hold onto the platform 225 and comfortably operate both pincer sets individually using different fingers of one hand. With the configuration as shown in FIG. 10, a surgeon may be capable of grasping three different surgical sites at different times or simultaneously to more precisely perform a surgical operation. Of course, the present invention is not limited thereto, as an increased number of pincer sets and varied pincer set formation may be attached to the platform.

Furthermore, according to another exemplary embodiment of the present invention, a method for grasping may include providing a medical device that has a platform and a plurality of pincer sets attached to the platform and extending from the platform at different lengths. The pincer sets may be a pair of tweezers, forceps, clips, or other similar mechanism. In addition, the method may include individually operating the plurality of pincer sets with one hand at a pair of mutually opposed arms of each pincer set and operating each pincer set disposed at different distances from the platform. A distance between each pincer set may be spread (e.g., increased, widened, etc.) by moving a fastener connected to the mutually opposed arms further from each pincer set.

The movement of the fastener spreads the pincer sets apart and such a distance may also be increased using a screw mechanism 510 attached to a back of the fastener, as previously discussed in relation to FIG. 5. In addition, the distance between each pincer set may be narrowed by moving the fastener connected to the mutually opposed arms toward each pincer set. Accordingly, the medical device may be used to grasp objects at different lengths and of different sizes. The medical device may be adjusted in varying methods such as disposing pincer sets at different lengths from the platform, increasing or decreasing the distance between pincer sets to be able to improve grasping precision while also operating a second medical device with a second hand.

Notably, although the above invention has been described in reference to typical surgical procedures and in relation to medical devices, the present invention is not limited thereto. The combined tweezers as disclosed herein may also be applied to other fields such as sewing, watch repairs, jewelry repairs or production, and other similar fields as understood by those of ordinary skill in the art.

What is claimed is:

1. A medical device comprising:
a platform; and
a plurality of pincer sets attached to the platform and extending from the platform at different lengths,
wherein the plurality of pincer sets are individually operable.

2. The medical device of claim 1, further comprising:
a holder attached to a bottom of the platform.

3. The medical device of claim 1, wherein the plurality of pincer sets are attached via a weld, a mold, an adhesive, a fastening mechanism, or a magnetic field.

4. The medical device of claim 1, wherein the plurality of pincer sets are selected from a group consisting of: tweezers, forceps, and clips.

5. The medical device of claim 1, wherein at least one set of pincers is operably connected to a second set of pincers.

6. The medical device of claim 1, wherein the platform is a flat, angled, spatial, or varied plane.

7. The medical device of claim 1, further comprising:
a rotational device attached to the platform and configured to move the plurality of pincer sets in multiple axis directions.

8. A medical device of claim 1, further comprising:
a pair of mutually opposed arms attached at a first end of each arm;
a first pair of pincers, separated by a first distance and extending from a second end of each arm; and
a second pair of pincers, separated by a second distance, with individual pincers attached to each mutually opposed arm,
wherein the first distance is different than the second distance.

9. The medical device of claim 8, wherein at least one of the first pair of pincers and the second pair of pincers is made of a resilient material.

10. The medical device of claim 8, wherein the second distance is less than the first distance.

11. The medical device of claim 8, further comprising:
a fastener connected to the mutually opposed arms and configured to spread or narrow the first distance and the second distance of the pincers.

12. A method for grasping, comprising:
providing a medical device having a platform and a plurality of pincer sets attached to the platform and extending from the platform at different lengths; and
individually operating the plurality of pincer sets with one hand at a pair of mutually opposed arms of each pincer set.

13. The method of claim 12, further comprising:
operating each pincer set disposed at different distances from the platform.

14. The method of claim 12, further comprising:
spreading a distance between each pincer set by moving a fastener connected to the mutually opposed arms further from each pincer set; and
narrowing the distance between each pincer set by moving the fastener connected to the mutually opposed arms toward each pincer set.

15. The method of claim 12, wherein the plurality of pincer sets are selected from a group consisting of: tweezers, forceps, and clips.
